(19) United States
(12) Patent Application Publication
(10) Pub. No.: US 2020/0397024 A1
(43) Pub. Date: Dec. 24, 2020

(54) MICROENCAPSULATED PRODUCTS, CLEAR SOLUTIONS THEREOF, AND METHODS OF MAKING

(71) Applicant: 463IP Partners, LLC, Grand Rapids, MI (US)

(72) Inventor: Gregory Thomas Grochoski, Ada, MI (US)

(21) Appl. No.: 16/976,924

(22) PCT Filed: Feb. 28, 2019
(86) PCT No.: PCT/US19/20107

§ 371 Date: Aug. 31, 2020

Related U.S. Application Data
(60) Provisional application No. 62/637,258, filed on Mar. 1, 2018.

(51) Int. Cl. A23L 2/395; A23L 27/00; A23P 10/30; A23L 29/25; A23L 29/30; A23L 3/46; A23L 33/175; A23L 33/115; A23L 2/56; A61K 45/06

(52) U.S. Cl. CPC A23L 2/395; A23L 27/72; A23P 10/30; A23L 29/25; A23L 29/35; A23V 2002/00; A23L 33/175; A23L 33/115; A23L 2/56; A61K 45/06; A23L 3/46

(57) ABSTRACT

Provided herein are methods for producing formulations for incorporation into clear beverages. A variety of solutes may be microencapsulated to provide a formulation that results in a clear solution upon addition to water. Microencapsulation may be achieved by, for example, spray drying. Also provided herein are microencapsulated products and solutions of microencapsulated products in water.

### BACKGROUND

It is well documented in food science that proteins, such as whey protein, milk protein, casein, soy protein, etc. and the range of amino acids which make up these proteins, disperse and dissolve in varying degrees of completeness in water and aqueous-based solvents (such as hydro-alcoholic solvents).

It is also well documented that these proteins and amino acids experience clumping and other limitations to readily being dispersed and/or solubilized. For this reason, processes known as “instantizing” are applied to these materials to improve their dispersibility and solubilization. The proteins or amino acids are supplied with small amounts of lecithin and spray dried. The resulting powder exhibits improved wetting (less clumping), dispersibility and rate of solubilization.

The hydrophilic tails of amino acids and amino acids comprising proteins facilitate their dispersion and dissolution in water.

Flavor oils added to protein and amino acid substrates are mostly hydrophobic and resist dispersion and dissolution in water.

Combining materials from these two classes of hydrophilic and hydrophobic characters into a single powder most commonly results in a difficult to disperse and/or cloudy solution.

Single amino acids, blends of amino acids, and blends of amino acids and other ingredients as well as other ingestible and injectable materials commonly form cloudy solutions when mixed in water or aqueous solutions (such as hydroalcoholic). Solution clarity provides a simple and effective means for consumers (users) to discern when a substance has been fully dissolved. Fully dissolved materials are generally known to be at their most absorbable and usable state. Additionally, solution clarity provides an effective means for consumers (users) to discern if an insoluble contaminant is present in the product. It has been discovered during a series of experiments that choice of source material and processing conditions can improve the clarity of solutions that are commonly cloudy and/or containing not fully dissolved solutes.

### SUMMARY

In one aspect of the invention, a method for producing a clear solution is provided, comprising: microencapsulating a solute in an encapsulant;
wherein the encapsulant comprises a first encapsulant material, or a second encapsulant material, or a combination thereof.

In another aspect of the invention, a microencapsulated product is provided, comprising: a solute microencapsulated in an encapsulant, the encapsulant comprising a first encapsulant material, a second encapsulant material, or a combination thereof.

In another aspect of the invention a solution of a microencapsulated product in water is provided.

### BRIEF DESCRIPTION OF THE DRAWINGS

FIG. 1 shows the clarity of a solution of a microencapsulated product of the present invention compared to the cloudiness of a solution of a non-microencapsulated product.

### DETAILED DESCRIPTION

Microencapsulation techniques are known in the art of food preparation. Provided herein is a means of selecting materials and distinct processing techniques to generate the uncommon result of a clear solution. Through a series of experiments, we were able to identify how to make compatible the competing characteristics of dispersion and solubilization of hydrophilic and hydrophobic materials in a single readily dispersible and soluble powder.

One aspect of the invention provides a method for creating clear solutions through ingredient screening and selection, and processing under specified processing conditions in spray drying, and/or blending, and/or fluid bed.

Microencapsulation via spray drying typically produces microencapsulates with insufficient solubility to form visually clear solutions when mixed in water or hydroalcoholic solvents. By selecting materials and processing conditions, clear solutions can be generated.

In one aspect, the invention provides a method for producing a clear solution, comprising:

microencapsulating a solute in an encapsulant;
wherein the encapsulant comprises a first encapsulant material, a second encapsulant material, or a combination thereof.

In some embodiments, the microencapsulating step is performed by spray drying.

In one embodiment, the first encapsulant material is a hydrocolloid gum. In some embodiments, the hydrocolloid gum is a powder. In some embodiments, a hydrocolloid gum is used as a wall material (encapsulating agent). In some embodiments, the hydrocolloid gum is gum Arabic, guar gum, gum Acacia, or xanthan gum. In some embodiments, the hydrocolloid gum is gum Arabic. In some embodiments, the hydrocolloid gum is gum Acacia. In some embodiments, the encapsulant comprises the hydrocolloid gum in an amount of from about 0 wt % to about 50 wt % (e.g., about 1 wt % to about 50 wt %, about 2 wt % to about 25 wt %, about 1 wt % to about 10 wt %, or about 5 wt % to about 15 wt %), by weight of the encapsulant.

In one embodiment, the second encapsulant material is a dextrin. In some embodiments the dextrin has a molecular weight of from about 504.5 g/mol to about 2774.7 g/mol. In some embodiments, the dextrin is a powder. In some embodiments, the dextrin is maltodextrin. In some embodiments the encapsulant comprises a dextrin (e.g., maltodextrin). In some embodiments the encapsulant comprises the dextrin (e.g., maltodextrin) in an amount of from about 5% to about 100% (e.g. about 5 wt % to about 95 wt %, about 5 wt % to about 50 wt %, about 5 wt % to about 25 wt %, about 5 wt % to about 15 wt %, or about 10 wt % to about 20 wt %), by weight of the encapsulant. In one embodiment, the second encapsulant material is a polysaccharide. In one embodiment, the second encapsulant material is a glycoprotein.

In some embodiments, the encapsulant comprises the first encapsulant material but not the second encapsulant material. In other embodiments, the encapsulant comprises the second encapsulant material but not the first encapsulant material. In some embodiments, the encapsulant comprises the first encapsulant material and the second encapsulant material. In some embodiments, the encapsulant is an encapsulant blend. The encapsulant blend may comprise the first encapsulant material with one or more additional ingredients, the second encapsulant material with one or more additional ingredients, the first encapsulant material and the second encapsulant material without additional ingredients, or the first encapsulant material and second encapsulant material with one or more additional ingredients.

In some embodiments, the encapsulant blend comprises the first encapsulant and the second encapsulant in a weight ratio of from about 0:100 to about 95:5, e.g., in some embodiments, from about 5:95 to about 95:5, from about 25:75 to about 75:25, from about 40:60 to about 60:40, from about 0:100 to about 20:80, about 50:50, or about 0:100.

In some embodiments, the encapsulant blend further comprises one or more flavors. In some embodiments, the encapsulant blend further comprises one or more flavor oils. In some embodiments, the flavors are selected from maltol, benzyl alcohol, butyl isovalerate, linalool, ethyl isovalerate, ethyl butyrate, gamma decalactone, anisyl acetone, beta ionone, or any combination thereof. In some embodiments, the encapsulant blend comprises from about 5 wt % to about 95 wt % of flavors (e.g., flavor oils) (e.g., about 50 wt % to about 90 wt %, about 70 wt % to about 90 wt %, or about 50 wt % to about 70 wt %) by weight of the encapsulant blend.

In some embodiments, the encapsulant and the encapsulant mixed with the solute (i.e., the “payload blend”) may exhibit a solubility of at least about 95% in water as determined by the Cano-Chauca method. Cano-Chauca, M, et al. Effect of carriers on the microstructure of mango powder obtained by spray drying and its functional characterization. Innovative Food Science and Emerging Technologies, v.6, n.4, p. 420-428, 2005. (1 g of powder stirred into 25 ml distilled water for 5 minutes with use of a blender. Centrifuge for 10 min at 3000×g. Dry 20 ml overnight at 105 degrees C. and weigh dried supernatant in relation to original powder weight).

In some embodiments, the spray drier inlet temperature is between about 135 and about 150° C.

In some embodiments, the spray drier flow rate is at least about 0.75 L/hour.

In some embodiments, the wall density value is between about 22% and about 25%.

The methods described herein may be used to provide a composition suitable for incorporation in a beverage. Incorporation of the produced composition in water or another solution results in a clear formulation. Multiple solutes may be incorporated in the microencapsulation process to provide a composition for incorporation in a dry beverage. The term solute is used to indicate that the material is capable of being dissolved, i.e., into a clear solution in water.

In some embodiments, the solute comprises an individual amino acid. In other embodiments, the solute comprises a blend of two or more amino acids. In some embodiments, the amino acids are essential amino acids. In some embodiments, the amino acids are selected from L-leucine, L-Lysine HCl, L-Valine, L-Isoleucine, L-Arginine, L-Threonine, L-Phenylalanine, L-Methionine, L-Histidine, L-Tryptophan, or any combination thereof. In some embodiments, the solute comprises from about 25 wt % to about 90 wt % of amino acids (e.g., about 40 wt % to about 80 wt %, or about 60 wt % to about 75 wt %) by weight of the solute. In another embodiment, the solute comprises one or more amino acids and other dietary ingredients such as, but not limited to, Vitamin D, Caffeine, L-Glutamine. In other embodiments, the solute comprises one or more amino acids and other food ingredients such as proteins, carbohydrates and/or fats. In other embodiments, the solute comprises one or more amino acids and one or more drug ingredients. In other embodiments, the solute comprises one or more food ingredients. In other embodiments, the solute comprises one or more dietary supplement ingredients. In other embodiments, the solute comprises one or more drug ingredients. In other embodiments, the solute comprises one or more animal food ingredients. In other embodiments, the solute comprises one or more animal drug ingredients. In some embodiments, the solute is a powder.

In some embodiments, the solute further comprises soluble corn fiber. In some embodiments, the solute comprises from about 2 wt % to about 25 wt % soluble corn fiber (e.g., about 5 wt % to about 20 wt %, about 5 wt % to about 15 wt %, or about 10 wt %) by weight of the solute.

In some embodiments, the solute further comprises one or more of citric acid, malic acid, sodium citrate, sucralose, and acesulfame potassium.

In some embodiments the weight ratio of solute:encapsulant is from about 20:1 to about 1:1 (e.g., about 15:1 to about 2:1, about 15:1 to about 5:1, about 10:1 to about 2:1, about 10:1 to about 5:1, or about 12:1 to about 4:1).

In embodiments where the solute is an amino acid, the amino acid may be spray dried prior to microencapsulation. In some embodiments, the solute is a blend of amino acids and some of the individual amino acids are spray dried prior to microencapsulation.

In another aspect of the invention, a microencapsulated product is provided, comprising: a solute microencapsulated in a first encapsulant material, or a second encapsulant material, or a combination thereof.

As a person of ordinary skill in the art will readily understand, the microencapsulated product may comprise any of the materials, in any of the amounts, and may be produced according to any of the processes described above. Likewise, the materials, amounts, and structure of the products described below may be produced by a method described above.

In one embodiment, the microencapsulated product comprises a core comprising a solute (e.g., as described above) and a shell comprising a first encapsulant material, or a second encapsulant material, or a combination thereof (e.g., as described above).

In some embodiments, the microencapsulated product may exhibit a solubility of at least about 95% in water as determined by the Cano-Chauca method.

In some embodiments, the microencapsulated product comprises: a solute microencapsulated in an encapsulant, wherein:

the solute comprises one or more amino acids; and

the encapsulant comprises a hydrocolloid gum, a dextrin, or a combination thereof.

In some embodiments, the solute of the microencapsulated product further comprises soluble corn fiber.

In some embodiments, the encapsulant of the microencapsulated product further comprises one or more flavors, e.g., flavor oils.

In one embodiment, the microencapsulated product comprises: a solute microencapsulated in an encapsulant, wherein:
the solute comprises from about 40 wt % to about 90 wt % of total amino acid content by weight of the solute, and from about 5 wt % to about 25 wt % of soluble corn fiber by weight of the solute; and
the encapsulant comprises from about 50 wt % to about 90 wt % of total flavor content by weight of the encapsulant, and from about 10 wt % to about 50 wt % of a dextrin, a hydrocolloid gum, or a combination thereof, by weight of the encapsulant.

In some embodiments the microencapsulated product has a weight ratio of solute:encapsulant of from about 20:1 to about 1:1 (e.g., about 12:1 to about 4:1)

In some embodiments, the solute of the microencapsulated product comprises from about 50 wt % to about 80 wt % of total amino acid content by weight of the solute, and from about 5 wt % to about 15 wt % of soluble corn fiber by weight of the solute.

In some embodiments, the encapsulant of the microencapsulated product comprises from about 70 wt % to about 90 wt % of total flavor content by weight of the encapsulant, and from about 10 wt % to about 30 wt % of a dextrin, a hydrocolloid gum, or a combination thereof, by weight of the encapsulant.

In some embodiments, the encapsulant of the microencapsulated product comprises from about 70 wt % to about 90 wt % of total flavor oil content by weight of the encapsulant, and from about 10 wt % to about 30 wt % of maltodextrin by weight of the encapsulant.

In one embodiment, the microencapsulated product comprises: a solute microencapsulated in an encapsulant, wherein:
the solute comprises one or more amino acids and soluble corn fiber; and
the encapsulant comprises one or more flavors, and (i) a dextrin, (ii) a hydrocolloid gum, or (iii) a combination thereof;
wherein the microencapsulated product comprises by weight of the microencapsulated product:
   from about 30 wt % to about 80 wt % of total amino acid content;
   from about 4 wt % to about 20 wt % soluble corn fiber;
   from about 5 wt % to about 15 wt % of total flavor oil content; and
   from about 0.5 wt % to about 5 wt % of a dextrin, a hydrocolloid gum, or a combination thereof.

In some embodiments, the microencapsulated product comprises by weight of the microencapsulated product:
from about 50 wt % to about 70 wt % of total amino acid content;
from about 5 wt % to about 12 wt % soluble corn fiber;
from about 6 wt % to about 13 wt % of total flavor oil content; and
from about 1 wt % to about 3 wt % of a dextrin, a hydrocolloid gum, or a combination thereof.

In some embodiments, the microencapsulated product comprises by weight of the microencapsulated product: from about 1 wt % to about 3 wt % of maltodextrin.

In another aspect of the invention a solution of a microencapsulated product in water is provided.

In some embodiments, the microencapsulated product is at least about 95% soluble in the water as determined by the Cano-Chauca method.

### EXAMPLES

### Example 1. Example Procedure for Preparing Formulations for Clear Solutions

An encapsulant blend was prepared by mixing gum Arabic and maltodextrin in water. A solute was progressively added to the encapsulant blend while stirring to produce a payload blend. The relative concentrations of the payload blend was varied depending on the properties of the solute. The payload blend was used as the feeding liquid for spray drying.

The payload blends were dried using a spray drier. Inlet air temperatures were varied according to the properties of the payload blends. Feed flow rates were varied according to the properties of the payload blends. Upon spray-drying a microencapsulated product was formed.

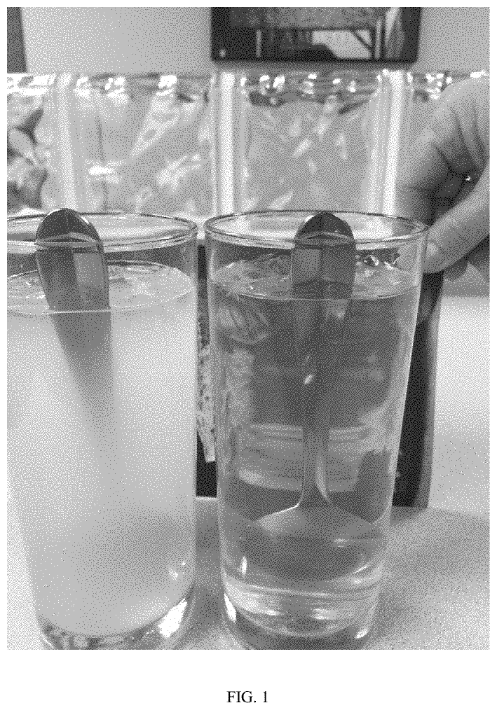

The resulting microencapsulated products were unexpectedly clear upon addition to water. A microencapsulated product that was an embodiment of the present invention was prepared according to Example 1 and was added to a glass of water. A non-microencapsulated product was added to a glass of water as a comparison. As shown in FIG. 1, the microencapsulated product had significantly better clarity and solubility than the non-microencapsulated product.

Examples of formulas made using methods of the described invention are provided in Examples 2-5 below.

### Example 2. Composition of Frist Brazilian Berry Formulation

| | Amount | % of |
|---|---|---|
| Ingredient | (mg) | formulation |
| Amino Acid Blend | 3904 | 61% |
| (L-leucine, L-Lysine HCl, | | |
| L-Valine, L-Isoleucine, | | |
| L-Arginine, L-Threonine, | | |
| L-Phenylalanine, L-Methonine, | | |
| L-Histidine, and | | |
| L-Tryptophan) | | |
| Citric Acid | 800 | 12.5% |
| Encapsulant Blend (2A or 2B) | 700 | 10.9% |
| Soluble Corn Fiber | 566 | 8.8% |
| Malic Acid | 170 | 2.7% |
| Sodium Citrate | 150 | 2.3% |
| Sucralose | 80 | 1.3% |
| Acesulfame Potassium | 30 | 0.5% |

### Example 2A. First Encapsulant Blend Formulation

| | Amount | % of |
|---|---|---|
| Ingredient | (mg) | formulation |
| Flavor (maltol, benzyl alcohol, | 595 | 85% |
| butyl isovalerate, linalool, | | |
| ethyl isovalerate, ethyl | | |
| butyrate, gamma decalactone, | | |
| anisyl acetone, and | | |
| beta ionone) | | |
| Maltodextrin | 105 | 15% |

### Example 2B. Second Encapsulant Blend Formulation

| | Amount | % of |
|---|---|---|
| Ingredient | (mg) | formulation |
| Flavor (maltol, benzyl alcohol, | 560 | 80% |
| butyl isovalerate, linalool, | | |
| ethyl isovalerate, ethyl | | |
| butyrate, gamma decalactone, | | |
| anisyl acetone, | | |
| and beta ionone) | | |
| Maltodextrin | 70 | 10% |
| Gum Acacia | 70 | 10% |

### Example 3. Composition of Second Brazilian Berry Formulation

| | Amount | % of |
|---|---|---|
| Ingredient | (g) | formulation |
| Reginator ® V amino | 3.7900 | 60.9953 |
| acid blend | | |
| Sucralose | 0.0800 | 1.2500 |
| Acesulfame Potassium | 0.0300 | 0.4688 |
| Citric Acid Anhyd. | 0.8000 | 12.5000 |
| Fine Gran. | | |
| DL-Malic Acid | 0.1700 | 2.6563 |
| Fine Gran. | | |
| Sodium Citrate | 0.1500 | 2.3438 |
| Dihydrate | | |
| Nat. Bitter Blocker | 0.3000 | 4.6875 |
| Fibersol-2 Non-GMO | 0.5663 | 8.8484 |
| Wildberry Nat. | 0.4000 | 6.2500 |
| Type Flavor | | |

### Example 4. Composition of Fuji Grape Formulation

| | Amount | % of |
|---|---|---|
| Ingredient | (g) | formulation |
| Reginator ® V amino | 3.7900 | 63.9950 |
| acid blend | | |
| Sucralose | 0.0800 | 1.3115 |
| Acesulfame Potassium | 0.0300 | 0.4918 |
| Citric Acid Anhyd. | 0.3600 | 5.9016 |
| Fine Gran. | | |
| DL-Malic Acid | 0.1200 | 1.9672 |
| Fine Gran. | | |
| Tartaric Acid | 0.3000 | 4.9180 |
| Sodium Citrate | 0.1500 | 2.4590 |
| Dihydrate | | |
| Nat. Bitter Blocker | 0.3000 | 4.9180 |
| Fibersol-2 Non-GMO | 0.5663 | 9.1197 |
| Nat. Concord Grape | 0.3000 | 4.9180 |
| Type Flavor | | |

### Example 5. Composition of Kona Dragon Fruit formulation

| | Amount | % of |
|---|---|---|
| Ingredient | (g) | formulation |
| Reginator ® V amino | 3.7900 | 63.9950 |
| acid blend | | |
| Sucralose | 0.0800 | 1.3115 |
| Acesulfame Potassium | 0.0300 | 0.4918 |
| Citric Acid Anhyd. | 0.8000 | 13.1148 |
| Fine Gran. | | |
| DL-Malic Acid | 0.1700 | 2.7869 |
| Fine Gran. | | |
| Sodium Citrate | 0.1500 | 2.4590 |
| Dihydrate | | |
| Nat. Bitter Blocker | 0.3000 | 4.9180 |
| Fibersol-2 Non-GMO | 0.5683 | 9.3164 |
| Black Cherry | 0.0980 | 1.6066 |
| Dragonfruit | | |

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

What is claimed is:

1. A method for producing a clear solution, comprising:
microencapsulating a solute in an encapsulant;
wherein the encapsulant comprises a first encapsulant material, a second encapsulant material, or a combination thereof.

2. The method of claim 1, wherein the microencapsulating step is performed by spray drying.

3. The method of claim 1, wherein the first encapsulant material is a hydrocolloid gum.

4. The method of claim 1, wherein the first encapsulant material is gum Arabic or gum Acacia.

5. The method of claim 1, wherein the second encapsulant material is a dextrin.

6. The method of claim 1, wherein the second encapsulant material is maltodextrin.

7. The method of claim 1, wherein the encapsulant comprises from about 10 wt % to about 50 wt % of the first encapsulant material, second encapsulant material, or combination thereof, by weight of the encapsulant.

8. The method of claim 1, wherein the encapsulant has a solubility of at least about 95% in water as determined by a Cano-Chauca method.

9. The method of claim 2, wherein the spray drying is performed by a spray drier having a spray drier inlet, and the spray drier inlet has a temperature of from about 135 to about 150° C.

10. The method of claim 2, wherein the spray drying is performed by a spray drier having a spray drier flow rate, and the spray drier flow rate is at least about 0.75 L/h.

11. The method of claim 1, wherein the encapsulant has a wall density value, and the wall density value is from about 22% to about 25%.

12. The method of claim 1, wherein the solute comprises individual amino acids.

13. The method of claim 1, wherein the encapsulant does not comprise lecithin.

14. The method of claim 1, wherein the solute comprises a blend of two or more amino acids.

15. The method of claim 1, wherein the solute comprises one or more amino acids and other dietary ingredients such as, but not limited to, Vitamin D, Caffeine, L-Glutamine, etc.

16. The method of claim 1, wherein the solute comprises one or more amino acids and other food ingredients such as proteins, carbohydrates and/or fats.

17. The method of claim 1, wherein the solute comprises one or more amino acids and one or more drug ingredients.

18. The method of claim 1, wherein the solute comprises one or more food ingredients.

19. The method of claim 1, wherein the solute comprises one or more dietary supplement ingredients.

20. The method of claim 1, wherein the solute comprises one or more drug ingredients.

21. The method of claim 1, wherein the solute comprises one or more animal food ingredients.

22. The method of claim 1, wherein the solute comprises one or more animal drug ingredients.

23. A microencapsulated product comprising a solute microencapsulated in an encapsulant, wherein the encapsulant comprises a first encapsulant material, or a second encapsulant material, or a combination thereof.

24. The product of claim 23, wherein the first encapsulant material is a hydrocolloid gum.

25. The product of claim 24, wherein the first encapsulant material is gum Arabic or gum Acacia.

26. The product of claim 23, wherein the second encapsulant material is dextrin.

27. The product of claim 26, wherein the second encapsulant material is maltodextrin.

28. The product of any one of claims 23-27, wherein the encapsulant further comprises one or more flavors.

29. The product of claim 28, wherein the one or more flavors comprise one or more flavor oils.

30. The product of any one of claims 23-29, wherein the solute comprises one or more amino acids.

31. The product of claim 30, wherein the solute comprises one or more essential amino acids.

32. The product of claim 30 or 31, wherein the solute further comprises soluble corn fiber.

33. The product of claim 23, wherein the encapsulant comprises maltodextrin.

34. The product of claim 33, wherein the encapsulant further comprises gum Arabic or gum Acacia.

35. The product of claim 23, wherein:
the solute comprises from about 40 wt % to about 90 wt % of total amino acid content by weight of the solute, and from about 5 wt % to about 25 wt % of soluble corn fiber by weight of the solute; and
the encapsulant comprises from about 50 wt % to about 90 wt % of total flavor content by weight of the encapsulant, and from about 10 wt % to about 50 wt % of a dextrin, a hydrocolloid gum, or a combination thereof, by weight of the encapsulant.

36. The product of claim 35, wherein:
the solute of the microencapsulated product comprises from about 50 wt % to about 80 wt % of total amino acid content by weight of the solute, and from about 5 wt % to about 15 wt % of soluble corn fiber by weight of the solute; and
the encapsulant of the microencapsulated product comprises from about 70 wt % to about 90 wt % of total flavor content by weight of the encapsulant, and from about 10 wt % to about 30 wt % of a dextrin, a hydrocolloid gum, or a combination thereof, by weight of the encapsulant.

37. The product of claim 35 or 36, wherein the weight ratio of solute:encapsulant is from about 12:1 to about 4:1.

38. The product of claim 23, wherein:
the solute comprises one or more amino acids and soluble corn fiber; and
the encapsulant comprises one or more flavors, and (i) a dextrin, (ii) a hydrocolloid gum, or (iii) a combination thereof;
wherein the microencapsulated product comprises by weight of the microencapsulated product:
from about 30 wt % to about 80 wt % of total amino acid content;
from about 4 wt % to about 20 wt % soluble corn fiber;
from about 5 wt % to about 15 wt % of total flavor oil content; and
from about 0.5 wt % to about 5 wt % of a dextrin, a hydrocolloid gum, or a combination thereof.

39. The product of claim 38, wherein the microencapsulated product comprises by weight of the microencapsulated product:
from about 50 wt % to about 70 wt % of total amino acid content;
from about 5 wt % to about 12 wt % soluble corn fiber;
from about 6 wt % to about 13 wt % of total flavor oil content; and
from about 1 wt % to about 3 wt % of a dextrin, a hydrocolloid gum, or a combination thereof.

40. The product of any one of claims 23-39, wherein the microencapsulated product has a solubility of at least about 95% in water as determined by a Cano-Chauca method.
